(19) United States
(12) Patent Application Publication
(10) Pub. No.: US 2021/0145992 A1
(43) Pub. Date: May 20, 2021

(54) MEDICAL STERILIZER

(71) Applicant: CARELIVE CO., LTD., Bucheon-si (KR)

(72) Inventor: Hyun Sook KIM, Namyangju-si (KR)

(73) Assignee: CARELIVE CO., LTD., Bucheon-si (KR)

(21) Appl. No.: 16/700,472

(22) Filed: Dec. 2, 2019

(30) Foreign Application Priority Data
Nov. 19, 2019 (KR) 10-2019-0148545

(51) Int. Cl. A61L 2/10

(52) U.S. Cl. CPC A61L 2/10; A61L 2202/24; A61L 2202/122

(57) ABSTRACT

A medical sterilizer for sterilizing a dental handpiece is disclosed. The medical sterilizer according to an embodiment of the present invention comprises: a transparent case made of a transparent material through which the interior can be seen and constitutes a part of the body; an opaque case formed by extending upwards of the transparent case, having a light source inside the upper end that radiates ultraviolet rays and is made of opaque material that does not allow the ultraviolet rays radiating from the above light source to pass through: a rubber stopper formed below the transparent case and containing a hole in which the dental handpiece can be inserted; and a lower portion fixing cap securing the rubber stopper to the opaque case.

### CROSS-REFERENCE TO RELATED PATENT APPLICATION

This U.S. non-provisional patent application claims priority under 35 U.S.C. § 119 of Korean Patent Application No. 10-2019-0148545 filed on Nov. 19, 2019 in the Korean Patent Office, the entire contents of which are hereby incorporated by reference.

### BACKGROUND

### 1. Technical Field

The present invention relates to a medical sterilizer. More particularly, the present invention relates to a medical sterilizer utilizing UV-C.

### 2. Description of the Related Art

The hospital is a place with facilities for diagnosing patients and treating patients according to the results of the diagnosis. Among many types of hospitals, dental clinics prevent, diagnose and treat diseases or abnormal conditions in the maxillofacial area including teeth and its surrounding tissues and oral cavity. Especially, attention to hygiene is needed because dental clinics have many tools that go into the patient's mouth and make direct contact with body fluids.

Dental clinics use dental care tools, commonly referred to as handpieces, to check or treat a patient's oral condition. In this case, the handpiece is equipped with a tool tip such as a bur and is used for culling the dentin of teeth or cleaning the treated area and collecting saliva, and the like.

Handpieces of the prior art have been stored mainly in the cradles of unit chairs combined with patient chairs and treatment tools. Such handpieces are more likely to get blood and/or saliva during the treatment of patients and therefore they must be sterilized in several stages after use.

However, the conventional method of sterilizing medical tools is equipped with a plasma or high pressure steam handpiece sterilization device in a separate sterilization place; the treatment tool must be transported separately from the air tube to a sterilization site equipped with a plasma or high pressure steam handpiece sterilization device; and the sterilizer must be replenished with purified water or gas. As a result, there is a problem that it takes an average of 30 minutes to 3 hours each time for sufficient sterilization through the above described process. Accordingly, the handpiece sterilization device of the prior art had a problem that it is difficult to sterilize the handpiece in real time before treating the patient.

### SUMMARY

### 1. Technical Problem

An object of the present invention is to suggest a medical sterilizer which can be inserted in a fixed position at all times when a dental handpiece is inserted.

### 2. Solution to Problem

A medical sterilizer for sterilizing a dental handpiece is disclosed. The medical sterilizer according to an embodiment of the present invention comprises: a transparent case made of a transparent material through which the interior can be seen and constitutes a part of the body; an opaque case formed by extending upwards of the transparent case, having a light source inside the upper end that radiates ultraviolet rays and is made of opaque material that does not allow the ultraviolet rays radiating from the above light source to pass through; a rubber stopper formed below the transparent case and containing a hole in which the dental handpiece can be inserted; and a lower portion fixing cap securing the rubber stopper to the opaque case.

### 3. Advantageous Effects

The medical sterilizer according to an embodiment of the present invention is provided with a rubber stopper for inserting a handpiece having a plurality of fixing pins and a rib structure, so that the handpiece can be fixed in the position having an optimum sterilization efficiency even when the handpiece is inserted from any direction.

### BRIEF DESCRIPTION OF DRAWINGS

FIG. 1 shows a medical sterilizer according to an embodiment of the present invention.

FIG. 2 is a cross-sectional view of a medical sterilizer according to an embodiment of the present invention.

FIGS. 3a-3d shows the detailed structure of a rubber stopper.

### DETAILED DESCRIPTION OF EMBODIMENTS

Hereinafter, a specific embodiment of the present invention will be described in detail with reference to the drawings. However, the spirit of the present invention is not limited to the following embodiments, and those skilled in the art who understand the spirit of the present invention may easily suggest by addition, alteration, deletion, and additional inclusion of other components that fall within the scope of the same spirit, but this is also included within the scope of the present invention.

The attached drawings may not express minute parts in detail in describing the overall structure in order to easily express the spirit of the present invention, and the overall structure may not be specifically reflected in describing the minute parts. In addition, when the action is the same, even if there is a specific difference in installation positions and the like, the same name is given, thereby improving the convenience of understanding. When there is a plurality of identical configurations, only one configuration will be described, and the same description will be applied to other configurations, and the description thereof will be omitted.

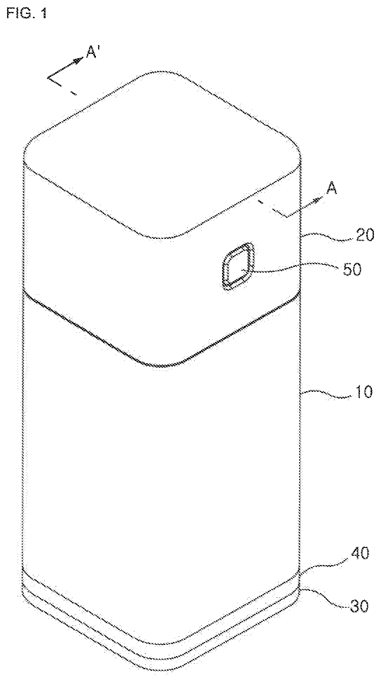

FIG. 1 shows a medical sterilizer according to an embodiment of the present invention.

FIG. 1 is an exemplary shape of a medical sterilizer according to an embodiment of the present invention, and it may have a different shape according to the use example. Hereinafter it will be described according to the exemplary shape of FIG. 1.

As illustrated in FIG. 1, a medical sterilizer 1 according to an embodiment of the present invention may comprise a transparent case 10, an opaque case 20, a lower portion fixing cap 30, a rubber stopper 40, and a switch 50.

The transparent case 10 constitutes a part of the body of the medical sterilizer 1. The transparent case 10 may be made of a transparent material through which the interior can be seen. The transparent case 10 may have a hexahedron shape.

The opaque case 20 together with the transparent case 10 constitutes a part of the body of the medical sterilizer 1. In other words, the transparent case 10 and the opaque case 20 may be referred to as a sterilizer body. The opaque case 20 may be made of an opaque material, and more particularly, may be made of an opaque material in which a light source that emits ultraviolet rays may not be directly exposed to a user.

The opaque case 20 may be provided on the transparent case 10. Specifically, the opaque case 20 is extended to have an outer surface of the same plane of the outer surface of the transparent case, and the upper end thereof may have a closed shape.

A switch 50 may be provided on one front surface of the opaque case 20. The switch 50 may be either a physical switch or a touch switch.

A rubber stopper 40 may be provided below the transparent case 10.

The rubber stopper 40 is a member for fixing the handpiece. In detail, the rubber stopper 40 may comprise a hole penetrating the inside and the outside of the medical sterilizer 1. In here, the hole may be a pathway through which the handpiece may enter or exit. In addition, the rubber stopper 40 may further comprise a handpiece fixing pin surrounding the hole. The specific features of the rubber stopper 40 will be described in more detail below.

The rubber stopper 40 may be fixed through the transparent case 10 and the lower fixing cap 30. Specifically, the rubber stopper 40 may comprise fixing holes near each corner, and the lower portion fixing cap 30 may fix the rubber stopper 40 to the body of the sterilizer through the fixing hole of the rubber stopper 40.

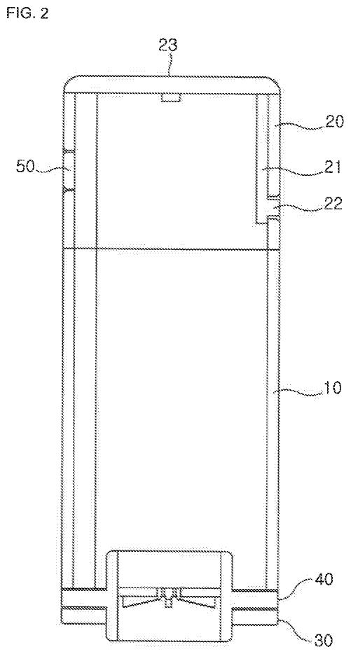

FIG. 2 is a cross-sectional view of a medical sterilizer according to an embodiment of the present invention. More specifically, FIG. 2 is a cross-sectional view illustrating a surface of the medical sterilizer cut along the line A-N of FIG. 1.

As illustrated in FIG. 2, an opaque case 20 is formed on top of the medical sterilizer 1, A transparent case 10 is formed below the opaque case 20. A rubber stopper 40 and a lower portion fixing cap 30 are formed below the transparent case 10.

A switch 50 may be formed on one surface of the opaque case 20. The switch 50 may be inserted into a groove formed on one surface of the opaque case 20.

A light source 23 may be formed on the inner upper surface of the opaque case 20. In a preferred embodiment, the light source 23 may be formed at the center of the upper end surface inside the opaque case 20. The light source 23 may be disposed to emit ultraviolet rays in the vertical downward direction.

The light source 23 is an ultraviolet C type (UV-C) light emitting diode (LED), and emits ultraviolet rays in a downward direction according to a switching signal by the switch 50. Here. UV-C type means the wavelength of 200 nm to 280 nm region in the wavelength range of UV LED. These UV-C type LEDs react with microorganisms of medical instruments with high energy and act as a disinfectant that destroys the DNA of the microorganisms. Herein, the amount of ultraviolet radiation of the light source 23 may be adjusted to have energy and time of a size capable of sterilizing all bacteria such as Escherichia coli. Pseudomonas aeruginosa, and mutants.

Another surface of the opaque case 20 may be formed with a groove for inserting a charging cable. A charging terminal 22 may be provided in the groove for inserting the charging cable.

In addition, a substrate and a battery 21 may be provided in the opaque case 20 by being electrically connected to the charging terminal 22. Specifically, the substrate and the battery 21 may be provided on one surface of the opaque case 20.

In the substrate and the battery 21, the substrate may comprise, for example, a printed circuit board and a microchip and one or more battery cells and may comprise a configuration for other electrical control.

In a specific embodiment, the battery cell may be connected to the charging cable to store power, and output power in accordance with the on/off operation of the switch 50. The printed circuit board and the microchip perform power control commands according to the on/off operation of the switch 50 to apply power to the light source.

As a result, the switch 50, the light source 23, the substrate, and the battery 21 are electrically connected, and the specific connection method follows a method known in the art.

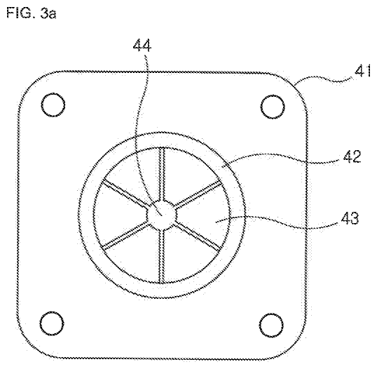

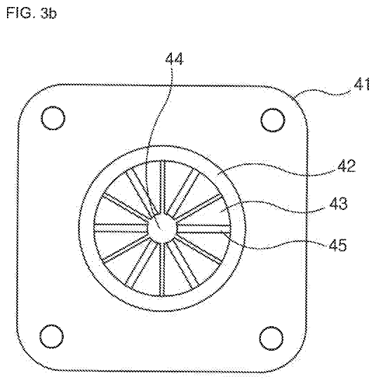

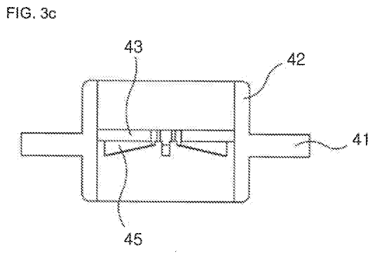

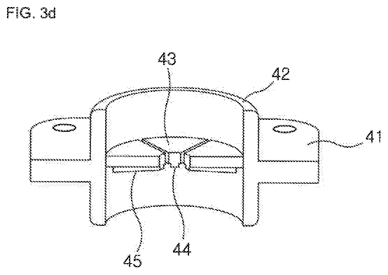

A rubber stopper 40 is formed at the bottom of the transparent case. The handpiece is inserted into the medical sterilizer 1 through the rubber stopper 40, and at the same time, the handpiece may be supported by the rubber stopper 40 and fixed to the center of the medical sterilizer 1. Hereinafter, the detailed structure of the rubber stopper 40 assisting the handpiece inserted into the medical sterilizer 1 to be fixed at a specific position will be described in FIG. 3.

FIG. 3 shows the detailed structure of the rubber stopper.

FIG. 3A is a plan view of the rubber stopper 40, FIG. 3B is a bottom view of the rubber stopper 40, FIG. 3C is a cross-sectional view of the rubber stopper 40, and FIG. 3D is a cross-sectional perspective view of the rubber stopper 40.

FIG. 3a will be described first. As illustrated in FIG. 3a, the rubber stopper 40 comprise, a fixing portion 41 forming an outer periphery, a guide 42 formed in the shape of a cylinder at the inner side of the fixing portion 41, and a handpiece fixing pin 43 formed in a predetermined number by being extended inwardly of the guide 42 and.

One end of the handpiece fixing pin 43 has a concave shape, and multiple one end of the handpiece fixing pins 43 are gathered to form a central hole 44 of a circular shape.

The fixing portion 41 is a member for fixing the rubber stopper 40 between the transparent case 10 and the lower portion fixing cap 30. A part of the outer portion of the fixing portion 41 may be fixed between the transparent case 10 and the lower portion fixing cap 30.

In addition, the fixing portion 41 may comprise a fixing hole as illustrated in FIG. 3. Fixing pins formed in the lower portion fixing cap 30 may be passed through the fixing hole. The fixing pin formed on the lower portion fixing cap 30 may be inserted into the transparent case 10 and fixed thereto. As a result, the rubber stopper 40 is fixed in the vertical direction through a part of the outer portion of the fixing portion 41, and may be fixed in the left and right directions through the fixing hole.

The guide 42 is formed inward of the fixing portion 41 and has a cylindrical shape. The top/bottom of the guide 42 is open.

The handpiece fixing pin 43 is formed on the inner side surface of the guide 42 and is formed to face the inner center of the guide 42. The handpiece fixing pin 43 may be formed in plural. In a preferred embodiment, the number of handpieces fixing pin 43 may be four or six.

And the end of the handpiece fixing pin 43 may have a concave shape. Therefore, a plurality of concave handpiece fixing pins 43 may be gathered to form a central hole 44 of a circular shape.

At this time, the diameter of the central hole 44 formed by the ends of the handpiece fixing pins 43 may be determined according to the diameter of the handpiece to be inserted. In a specific embodiment, the diameter of the central hole 44 formed by the ends of the handpiece fixing pins 43 may be determined to have an appropriate fitting tolerance value with the handpiece diameter.

The central hole 44 guides the handpiece to be easily inserted into a medical sterilizer 1. Without the central hole 44, the user may need more force to insert the handpiece into the medical sterilizer 1 and may not be able to accurately recognize the insertion position.

And, as illustrated in FIG. 3b, a rib structure 45 may be formed at the lower end of the handpiece fixing pin 43. Specifically, a rib structure 45 in the shape of a straight line may be formed in one direction from one end of the guide 42 to the other end of the guide 42 at the center of the lower end surface of the handpiece fixing pin 43.

The rib structure 45 is formed at the lower end of the handpiece fixing pin 43, as illustrated in FIGS. 3c and 3d. One end of the rib structure 45 may be in the shape of a slanted surface that is getting lowered as it travels toward the central hole 44 from the direction of the guide 42.

A medical sterilizer 1 according to an embodiment of the present invention, even if the handpiece is inserted from any direction through the rib structure 45 in the shape of a slanted surface, the end of the handpiece may be guided toward the center of the upper end of the medical sterilizer 1. Specifically, since the size of the central hole 44 is formed to have an appropriate fitting tolerance value with the diameter of the handpiece, the handpiece can be fixed when the handpiece is inserted.

And, since the rib structure 45 supports the handpiece guide pin 43 from below, the handpiece guide pin 43 can be maintained horizontally. And the end of the handpiece inserted by the plurality of handpiece guide pins 43 which is maintained horizontally is guided to face toward the center of the upper end of the medical sterilizer 1.

If the handpiece guide pin 43 does not have the rib structure 45, the handpiece guide pins 43 cannot be maintained horizontally in the situation where the handpiece is inserted, and each of the handpiece guide pins 43 may be directed at an angle different from others or bent. In this case, since the support by the handpiece guide pin 43 is not constant, there is a high possibility that the end of the handpiece may not face the center of the upper end of the inside of the medical sterilizer 1.

At the center of the upper end of the medical sterilizer 1, a light source 23 that emits ultraviolet rays is located, since the handpiece should be placed in line with the light source 23 to maximize sterilization effect, it is very important to place one end of the handpiece in the center of the upper end of the inside the medical sterilizer 1. And since it is obvious that the user will not always insert the handpiece in a constant direction, but most will insert the handpiece at an oblique angle, in general, it is also difficult to expect the end of the handpiece to naturally direct the center of the upper end of the inside the medical sterilizer 1.

As a result, the medical sterilizer 1 according to an embodiment of the present invention uses the handpiece fixing pin 43 forming the central hole 44 and the rib structure 45 provided in the lower end of the handpiece fixing pin 43 so that even if the user inserts the handpiece at any angle, it can be guided to a position that maximizes the sterilization effect of the end of the handpiece. In here, the central hole 44 has a diameter corresponding to the thickness of the handpiece and may be located in line with the light source 23.

In a further embodiment, the position of the light source may be changed according to the embodiment, in this case the position of the central hole may be changed corresponding to the position of the light source. The position of the central hole can be changed by adjusting the size between the handpiece fixing pins. And the size of the rib structure of each of the handpiece fixing pins may also be changed by adjusting the size among the handpiece fixing pins.

The foregoing detailed description should not be construed as limiting in all respects but should be considered as illustrative. The scope of the present invention should be determined by reasonable interpretation of the appended claims, and all changes within the equivalent scope of the present invention are included in the scope of the present invention.

What is claimed is:

1. A medical sterilizer for sterilizing a dental handpiece comprising:
a transparent case made of a transparent material through which the interior can be seen and constitutes a part of the body;
an opaque case formed by extending upwards of the transparent case, having a light source inside the upper end that radiates ultraviolet rays and is made of opaque material that does not allow the ultraviolet rays radiating from the above light source to pass through;
a rubber stopper formed below the transparent case and containing a hole in which the dental handpiece can be inserted; and
a lower portion fixing cap securing the rubber stopper to the opaque case.

2. The medical sterilizer according to claim 1, wherein the rubber stopper comprises:
a fixing portion forming an outer periphery;
a guide formed in the shape of a cylinder at the inner side of the fixing portion;
a handpiece fixing pin formed in a plural number by being extended inwardly of the guide and; and
a rib structure formed in the shape of a straight line at the center of the lower end of the handpiece fixing pin.

3. The medical sterilizer according to claim 2, wherein the end of the handpiece fixing pin has a concave shape, and the concave shape of the end of a plurality of handpiece fixing pins is gathered to form a circular hole.

4. The medical sterilizer according to claim 3, wherein the circular hole is formed in line with the light source.

5. The medical sterilizer according to claim 2, wherein one end of the rib structure has the shape of a slanted surface which is getting lowered as it travels toward the opposite direction from the guide direction.

6. The medical sterilizer according to claim 1, wherein a switch is formed on one outer surface of the opaque case, and a substrate and a battery are formed at one surface of the inside of the opaque case, and wherein the switch, the light source, the substrate, and the battery are electrically connected.

7. The medical sterilizer according to claim 6, wherein the substrate comprises a printed circuit board and a microchip.

8. The medical sterilizer according to claim 1, wherein the light source is an LED of a UV-C type.
